(19) United States
(12) Patent Application Publication
(10) Pub. No.: US 2002/0006954 A1
(43) Pub. Date: Jan. 17, 2002

(54) Desmethyl tocopherols for preventing or slowing degenerative neurological diseases

(75) Inventors: Kenneth L. Hensley, Oklahoma City, OK (US); Robert A. Floyd, Oklahoma City, OK (US)

Correspondence Address: Daniel S. Hodgins, HEAD, JOHNSON & KACHIGIAN, 204 N. Robinson, Ste. 3030, Oklahoma City, OK, 73102, US

(73) Assignee: Oklahoma Medical Research Foundation

(21) Appl. No.: 09/794,293

(22) Filed: Feb. 27, 2001

Related U.S. Application Data
(60) Provisional application No. 60/186,456, filed on Mar. 2, 2000.

(51) Int. Cl. A61K 31/355

(52) U.S. Cl. 514/458

(57) ABSTRACT

The present invention involves the use of desmethyl tocopherols such as gamma tocopherol for the prevention of and treatment of neurological disorders. Dietary or parenteral administration of desmethyl tocopherols inhibits the undesired nitration of neurological components.

### CROSS-REFERENCE TO RELATED APPLICATIONS

Priority is claimed from provisional application U.S. Ser. No. 60/186,456 filed on Mar. 2, 2000, and incorporated by reference herein.

### STATEMENT REGARDING FEDERALLY SPONSORED RESEARCH OR DEVELOPMENT

The United States Government has rights to this invention insofar as it was supported in part by the National Institutes of Health (NS35747, PO1-AG05119 and 5P50-AG05144).

### BACKGROUND

The present invention relates to concentrated preparations of desmethyl tocopherols, including but not restricted to gamma tocopherol (γT), which localize to lipid environments in neural tissue and scavenge reactive species such as nitrogen species (RNS) by virtue of a phenolic structural element lacking one or more methyl substituents on the phenolic ring system. The capability to scavenge RNS imparts brain-protective and neuroprotective properties to the compound.

Tocopherols (Toc) are a class of lipophilic, phenolic compounds of plant origin. The major tocopherol found in mammalian tissue is alpha tocopherol (α-tocopherol, αT or vitamin E; FIG. 1), although significant quantities of demethylated (desmethyl) forms (particularly γ-tocopherol; FIG. 1) are also present. α-Tocopherol acts as a free radical scavenger (i.e., a chain-breaking antioxidant) when the phenolic head group encounters a free radical: Toc-OH+L.→Toc-O.+LH (Toc-OH=tocopherol, L.=lipid radical, LH=lipid)

The phenoxyl radical (Toc-.) is much more stable, and less reactive, than L.. The aromatic nature of the tocopherol ring system, combined with steric and electronic influences from the methyl substituents, stabilizes the tocopheroxyl radical and thereby ends the lipid peroxidation process. Eventually, Toc-.. is reduced back to Toc-OH by ascorbate acting in conjunction with NADPH reductase. While α-tocopherol is the major tocopherol in the body, other tocopherols exist. The second principal tocopherol in the human body is γ-tocopherol (γT), which, like α-tocopherol, is made by plants and taken into the human diet with foodstuffs.

Recently, it has become appreciated that reactive nitrogen species (RNS) are significant to many diseases including Alzheimer's disease (AD), amyotrophic lateral sclerosis (ALS), Parkinson's disease and other conditions where inflammatory reactions occur. RNS are derived from the enzymatic oxidation of arginine via the intermediate nitric oxide free radical (FIG. 2). Unlike oxygen-centered free radicals, reactive nitrogen species are not scavenged effectively by α-tocopherol. On the other hand, γ-tocopherol can react easily with RNS because of the presence of an open space on the chromanol head of the molecule (FIG. 1). The major product of γ-tocopherol reaction with RNS is 5-nitro-γ-tocopherol (5γT, FIG. 1). Recent discoveries indicate that (A) γT protects biological systems from RNS much more effectively than αT; (B) γT is extensively nitrated in the brain of Alzheimer's disease patients; (C) γT inhibits RNS toxicity to a critical enzyme (α-ketoglutarate dehydrogenase, or αKGDH) which is severely depleted in Alzheimer's disease; and (D) γT protects cultured brain cells from RNS. Thus, γT possesses unique biochemical functions as compared to αT that suggest γT may be a superior dietary supplement, neuroprotectant, or a preservative in systems exposed to RNS.

γ-Tocopherol (γT) is a natural product (a desmethyl tocopherol) of plant origin, present in many vegetable oils, especially soybean oil (1-2). γ-Tocopherol is normally taken into the body through consumption of foodstuffs. Human plasma γT concentration is variously reported as between 5 and 30% of that of alpha tocopherol (αT) (3). The γT/ αT ratio varies markedly among individuals; plasma γT/ αT proportionalities may be as low as 0.2% and as high as 30% (inventors' observations). αT and γT are absorbed equally well by the gut, but γT is packaged into lipoproteins less effectively than αT (4). Possibly for this reason, αT supplementation decreases systemic γT concentration (3-4).

To date, only three well-disseminated studies have compared αT and γT with respect to their ability to inhibit nitrative stress specifically (5-7). These studies generally investigated the in vitro reaction of nitrating equivalents with target substrates in “pure” chemical systems, and two of the three studies reached very different conclusions. The first investigation from Cooney's lab (5) reported that γT reaction with NO₂ gas was 6 times more rapid than the corresponding reaction of αT. Furthermore, exposure of αT (but not γT) to NO₂ caused the formation of a secondary nitrating species which could nitrate the target compound morpholine (5). In the same manuscript, Cooney et al. showed that γT was 4-fold more effective than αT at inhibiting neoplastic transformation of methylcholanthrene-treated C3H/10T½ fibroblasts, a process which the authors suggests might involve nitrative stress (5). The second study (Christen et al. 1997; reference 6) incorporated either αT or γT, or both, into liposomes which were then exposed to synthetic peroxynitrite (ONOO⁻). Christen and colleagues found that γT was twice as effective as αT at inhibiting lipid hydroperoxide formation in liposomes exposed to ONOO⁻. Moreover, these researchers found that γT nitration rates were not influenced by the presence of αT. This latter finding suggests that nitration of γT may occur preferentially to reaction with αT when both tocopherols are simultaneously exposed to a nitrating species. In the third study (7), Goss et al. take issue with the findings of Christen et al. and report that αT does spare γT in liposomes exposed to the superoxide and the NO-generating compound SIN- 1[5-amino-2-(4-morpholinyl)- 1,2,3-oxadiazolium].

A search of the literature revealed only two studies in which αT and γT were compared for efficacy using in vivo models of cardiovascular stress (no studies were found investigating neurological stress). In the first study (c. 1983), tocopherol-depleted rats were fed αT or γT for two weeks after chronic exposure to iron-dextran as an inducer of oxyradical stress (8). While both αT and γT inhibited systemic lipid oxidation in the animals, γT was approximately 35% as effective as αT. Lipid nitration was not an endpoint of this investigation, and physiologic parameters were not recorded. In a second and very recent study (9) rats on an otherwise normal diet were fed αT or γT (100 mg/kg/day) for 10 days after which the abdominal aorta was exposed to a patch soaked in 29% FeCl₃ (9). This stress induced obstructive thrombus within 20 minutes. Saldeen et al. found that γT supplementation was significantly more effective than αT supplementation at inhibiting iron-induced lipid peroxidation and occlusive thrombus (9). Time to occlusive thrombus was delayed by 25% in the αT-supplemented animals and by 65% in γT-supplemented animals (9). Platelet aggregation kinetics were similarly inhibited, with γT supplementation being 2-fold more efficacious than αT supplementation (9). Most importantly, the γT concentration in the plasma of the γT supplemented rats never exceeded 10% of the γT concentration although the feeding paradigm did increase γT levels 6-fold above baseline (9). By comparison, αT supplementation increased αT plasma concentration only 2-fold (9). When treatment effects were considered in reference to plasma tocopherol concentrations, the Saldeen study found γT to be 20-30 times more potent than αT at inhibition of throbogenic correlates. No conclusive explanation for the γT effect was offered by the Saldeen study, though superoxide dismutase activity increased significantly in the aortas of γT treated animals as compared to the αT treated group (9). The unexpected efficacy of γT might also stem from a differential vascular partitioning of γT, since γT is reportedly incorporated into endothelial cells more rapidly than is αT (10). In any case, the efficacy of γT as a vascular or neuroprotectant cannot be predicted from its bioactivity in traditional fertility assays, or from its oxyradical scavenging capacity as measured in vitro.

Several studies have indicated that protein nitration occurs in human neurodegenerative disease and in animal models. The present inventors have measured nitrated tyrosines (nitrotyrosine) in the Alzheimer's disease brain using instrumental methods (11); other researchers have measured protein nitration using antibody methods (12). Protein nitration also occurs in amyotrophic lateral sclerosis (ALS; see reference 13) and in animal models of Parkinson's disease (14). No published data indicates a consideration of desmethyl tocopherols as inhibitors of the underlying nitrative stress. No studies have been published where αT and γT (or other desmethyl tocopherols) were compared as neuroprotective agents. No data has yet been published to indicate specific depletion or nitration of desmethyl tocopherols in humans suffering from degenerative neurological disease or from animal models of the same. Alpha tocopherol has been evaluated for ability to slow the progression of Alzheimer's disease in one large-scale clinical trial (15) and future trials are planned (16); no trials have been announced to investigate γT. Effort is being put forth to increase αT levels in foodstuffs (17), while no attention has been given to γT. The concept that γT (or other desmethyl tocopherols) protect uniquely against RNS in degenerative neurological conditions is therefore a nonobvious advancement in the field of neurobiology and in the field of antioxidant therapeutics.

The present invention is intended to solve the problems described, namely, the inefficacy of alpha tocopherol (vitamin E) to adequately protect against nitration damage, and to improve the ability of the tocopherol to inhibit the progression of degenerative neurological diseases including but not limited to Alzheimer's disease.

### SUMMARY OF INVENTION

The present invention involves and discloses the use of gamma tocopherol and other desmethyl tocopherols as scavengers of reactive nitrogen or other species in tissue exposed to an inflammatory or other stress, specifically in brain tissue exposed to nitrative stress. The desmethyl tocopherols of the present invention have the following preferred structures:

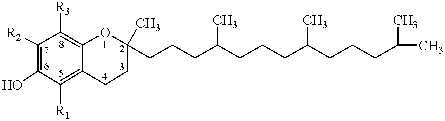

where at least one of R₁, R₂, and R₃ must be a H atom. Additionally, the alkyl tail of the molecule may contain either saturated or unsaturated variants (unsaturated variants including the chemical subclass of tocotrienol tocopherols). Since the main bioactive function of the above structure is the phenolic head group, any stereoisomer of the tocopherol may be used. Furthermore, since the main bioactive function of the above structure is the phenolic head group, any carbon can be eliminated from the carbon centers labeled 2-4 in the structure above. Furthermore, the —OH group can be esterified or otherwise modified to form a prodrug or a more water-soluble derivative such as an ester, which would regenerate the —OH group in vivo.

These and other homologs of the tocopherols can be chemically synthesized or isolated from natural sources. In the method of the present invention, the tocopherols are administered in a safe and effective amount to scavenge reactive nitrogen species and slow the progression of nitrative stress in tissue undergoing progressive degeneration. These and other advantages and objects of the invention will be apparent to those skilled in the art. While the mechanism of neurological protection by desmethyl tocopherols is believed known, there may be another or additional mechanism. That is the present invention is not shown by any single mechanism.

Subjects to be tested with desmethyl tocopherols include those with the early neurological symptoms well known to those skilled in the art as well as those having a familial history of neurological disease.

### BRIEF DESCRIPTION OF THE DRAWINGS

FIG. 1 illustrates tocopherol structures. The arrows indicate the 5 position of the chromanol ring system, which is methylated in α-tocopherol (vitamin E) but not in γ-Tocopherol.

FIG. 2 illustrates pathways for generation of nitrating agents and their subsequent reaction with phenolic substrates such as tyrosine or γ-tocopherol.

FIG. 3A is a HPLC-ECD chromatogram demonstrating detection of alpha, gamma and 5-nitro-gamma tocopherol in the Alzheimer's diseased brain. Key: a-α-tocopherol; b=γ-tocopherol; c=5-NO₂-γ-tocopherol.

FIG. 3B is a graph showing a regional variation in 5-NO₂-γ-tocopherol content demonstrating increased lipid nitration in cortical regions of the AD brain, but not in the cerebellum. *p<0.05; N=10-15. HIP=hippocampus; SMTG=superior and middle temporal gyrus; CBL=cerebellum.

FIG. 4 is a graph showing Nitrotyrosine and dityrosine in the AD brain. Analytes were determined in four regions of normal and Alzheimer's diseased brain (HIP=hippocampus; IPL=inferior parietal lobule; SMTG=superior and middle temporal gyri; CBL=cerebellum). N=5-10; *p<0.05. Note the relative sparing of the cerebellum.

FIG. 5 is a graph showing rat brain mitochondria were exposed to 0.4 mM SIN-1 for 1 H after addition of tocopherol. (.) α-toc; (▪) γ-toc. The scale bars labeled [α]ₙₒᵣₘ and [γ]ₙₒᵣₘ indicate the normal endogenous quantities of α-toc and γ-toc, respectively, in human brain.

FIG. 6A is a graph showing neurotoxicity induced by increasing concentrations of the NO-releasing agent S-nitrosoprusside (SNP). Cerebrocortical neurons were exposed to the indicated concentration of SNP for 24 hours. Viability was assessed by release of lactate dehydrogenase (LDH) by nonviable cells.

FIG. 6B is a graph showing tocopherol protection of cortical neurons from NO toxicity induced by SNP. Dashed line indicates % viability of cells treated with SNP only (no tocopherols). Control (no SNP)=100%. * p<0.05 relative to unprotected cells, N=5.

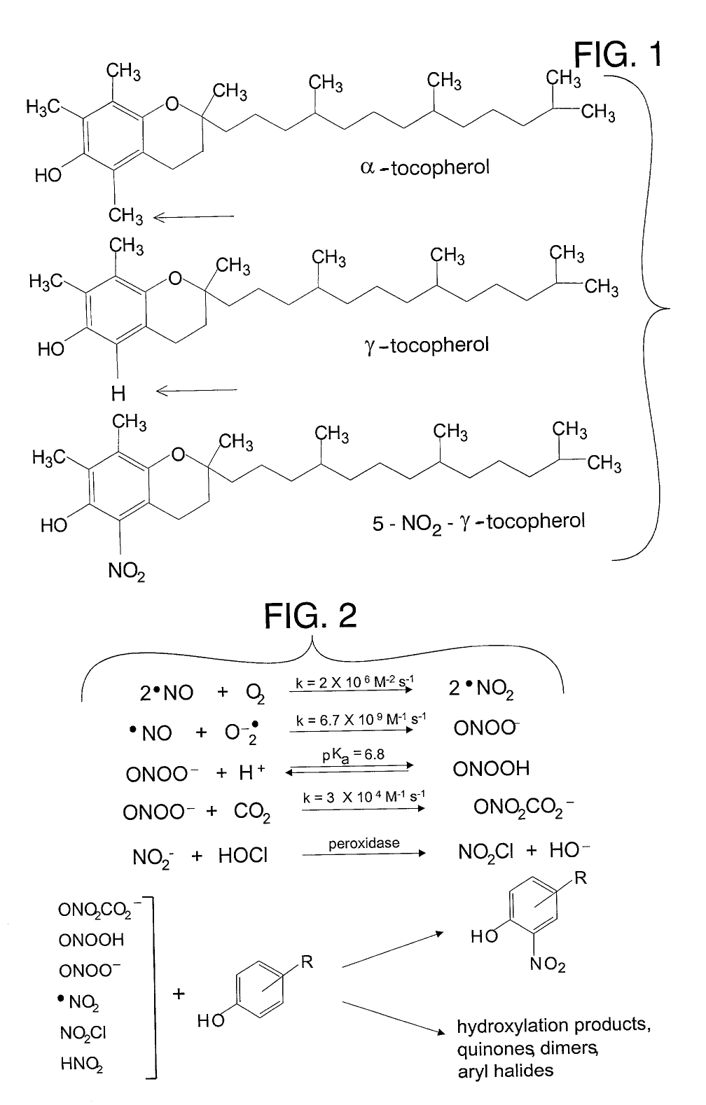

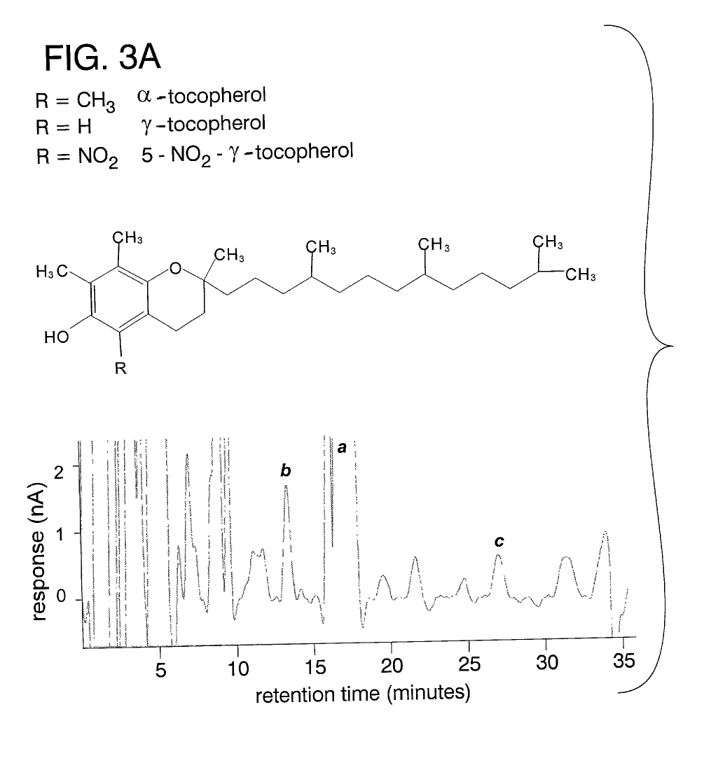

### DESCRIPTION OF PREFERRED EMBODIMENTS

The present application demonstrates the superiority of desmethyl tocopherols, exemplified by gamma tocopherol, as protectors against nitrative and/or other damage to biological systems. The results described here are novel in several respects. Particularly, the results demonstrate that gamma tocopherol (γ-tocopherol or γT) is superior to alpha tocopherol (i.e., vitamin E, a fully alkylated tocopherol) in systems where nitrative stress is a relevant phenomenon. The invention of this utility for γ-tocopherol (and other desmethyl tocopherols) is not obvious to most ordinarily skilled practitioners of the art of antioxidant therapy. This contention is demonstrated by the fact that only α-tocopherol is currently being studied as a clinically relevant antioxidant in the treatment of neurodegenerative disease (15). One study has found that α-tocopherol decreases the progression of Alzheimer's Disease (AD) marginally, and another large clinical trial is being planned (18); however, these plans only include the clinical assessment of α-tocopherol while desmethyl tocopherols are not being considered (18). Moreover, efforts are underway to create crop plants overproducing α-tocopherol at the expense of γ-tocopherol (17). In point of fact, oral supplementation of humans with α-tocopherol actually depletes the human body of γ-tocopherol (reference 3 and personal observations).

The γ-tocopherol and other desmethyl tocopherols are present in natural foods (particularly soy and wheat) in small amounts and are generally regarded as safe for human subjects. The biological activity of desmethyl tocopherols is associated with the chromanol head group of the molecule (indicated by arabic numbers in the structure above). This is to distinguish the tocopherols from tocotrienols, which inhibit cholesterol biosynthesis but whose activity is resident in the unsaturated lipid tail of the tocotrienol molecule. Gamma tocopherol (and other desmethyl tocopherols) may be chemically synthesized or isolated from natural products.

In practice, the γ-tocopherol (or other desmethyl tocopherols) would be formulated in a manner allowing safe delivery of effective doses to humans. The γ-tocopherol (or other desmethyl tocopherols) can be absorbed enterally by mammals and could be used by oral administration. The γ-tocopherol (or other desmethyl tocopherols) could be administered topically to inflamed skin or gum/mouth tissue as a cream or gel, or could be inhaled as an aerosol. The relative stability and lipophilicity of γ-tocopherol (and other desmethyl tocopherols) make these compounds amenable to delivery in numerous possible formulations. Derivatives of γ-tocopherol (or other desmethyl tocopherols) which retain the structure of a phenolic ring lacking a H atom near the —OH group would also be useful as protectant against nitrative stress in neurodegenerative conditions.

After consideration of the experimental data described below, these and other advantages and objects of the invention will be apparent to those skilled in the art.

As a cardioprotectant or neuroprotectant, oral γ-tocopherol supplements are taken at a dose of 100-400 mg/day by individuals suffering from or at risk for these diseases. The γ-tocopherol supplements would consist of γ-tocopherol alone or as a predominant component mixed with other tocopherols, mediations or nutritive supplements. As a component of topical products or for intravenous administration, γ-tocopherol could be used alone or in combination with α-ketoglutarate and other tocopherols. In these applications, effective concentrations would likely be from about 0.1 μM to about 10 mM, more preferably from about 0.1 mM to about 10 mM. While the above enteral administration is preferred, parenteral administration may be desirable to more rapidly and consistently elevate γT levels or to more directly locate more optimal dosages.

The following examples are intended for illustrative purpose only and are not to be construed as limiting the invention in spirit or scope.

### EXAMPLE 1

Demonstration that γ-Tocopherol Scavenges RNS (reactive nitrogen species) in the Alzheimer's Diseased Brain

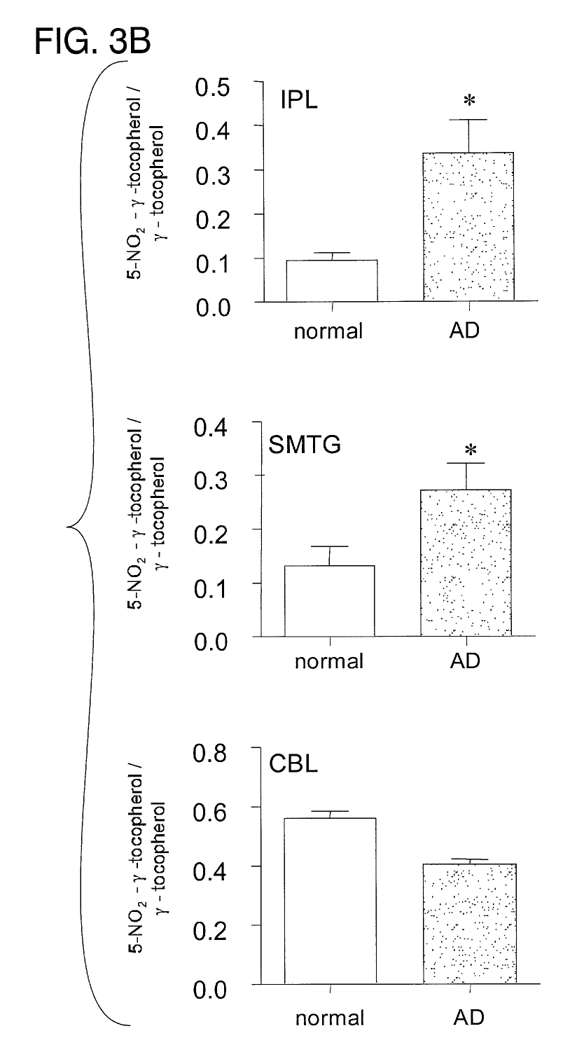

To study lipid-phase nitration chemistry, high performance liquid chromatography with electrochemical detection (HPLC-ECD) has been applied by the present inventors to the study of tocopherol variants and their oxidation products (18). By connecting a photodiode array detector in-line with (preceding) the ECD, at least 7 discreet tocopherol variants can be simultaneously quantified in hexane-extracted human plasma (18). In studies with brain tissue taken from Alzheimer's diseased (AD) and normal brains by rapid postmortem protocol (<3 H), a significant 2-3 fold increase can be seen in the 5-nitro-γ-tocopherol γ-tocopherol ratio in affected regions of the Alzheimer's diseased brain relative to age-matched normal brain (FIG. 3A and 3B). Concomitantly, the γT content of the cortical tissue decreased by 20-50% in AD depending on the brain region analyzed. The cerebellum, which is not severely affected in AD, was not severely nitrated (FIG. 3B). In a partial subcellular fractionation of these human brains, we found the highest level of tocopherol nitration in the mitochondrial fraction, where up to ⅔ of the γT appears to be nitrated. Moreover, the 5NγT content of human brain mitochondria (normal and AD combined) correlates inversely \hat{R}=−0.41; p<0.05; not illustrated) with the marker enzyme α-ketoglutarate dehydrogenase (αKGDH), perhaps indicating that nitrative stress is a significant factor in age-related mitochondrial dysfunction. Importantly, no significant decrease in α-tocopherol was observed in these studies, despite the profligate nitration of γ-tocopherol.

### EXAMPLE 2

Demonstration that Protein Nitration Occurs in the Alzheimer's Diseased Brain

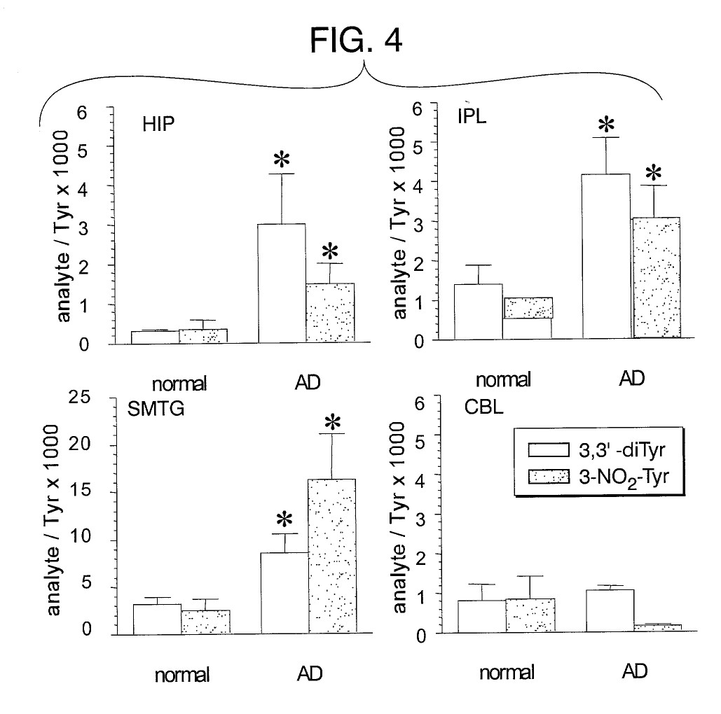

Using HPLC-ECD techniques, we measured 2-7 fold increases in protein nitration and oxidation products in the Alzheimer's brain and published these results (11). As illustrated in FIG. 4, nitrotyrosine (3-NO₂-Tyr) was significantly elevated in regions of the Alzheimer's diseased brain that are histologically affected by the disease.

### EXAMPLE 3

Demonstration of αKGDH Protection Against Nitrative Stress by γ-Tocopherol

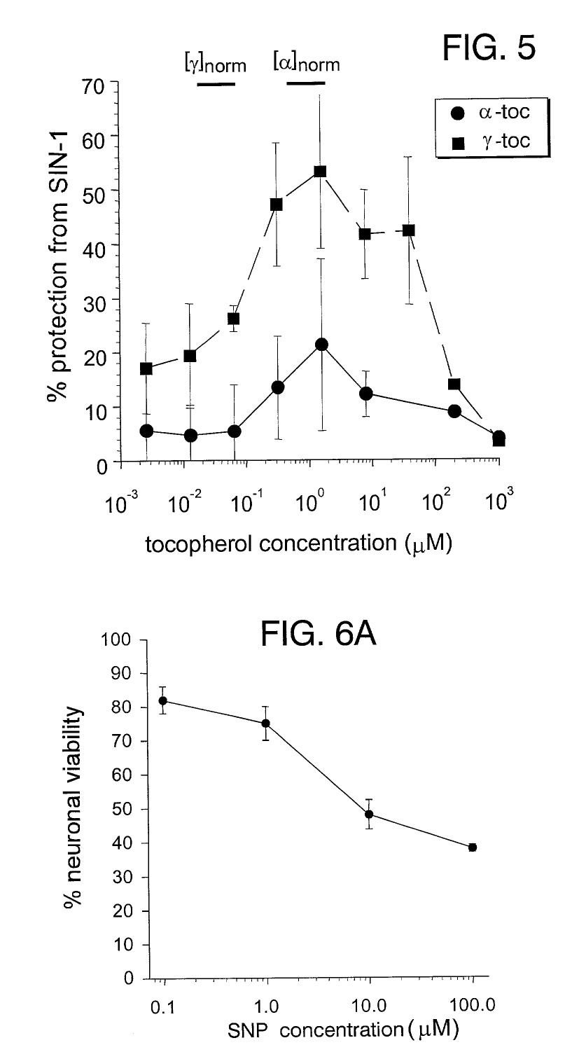

The finding that human brain αKGDH activity correlates negatively with mitochondrial 5NγT stimulated us to ask whether γT could protect mitochondria from nitrative stress in vitro. Mitochondria were isolated from adult rat brain then sonicated briefly in the presence of either αT or γT, or an ethanol vehicle. Mitochondria were then exposed to SIN-1, which generates NO and superoxide simultaneously at a known rate (7). Combination of NO and superoxide yields ONOO⁻ in situ (discussed above). Enzyme activity is destroyed by peroxynitrite and superoxide but not by NO (19). Predictably, αKGDH activity is diminished in lipopolysaccharide-treated cell culture in an RNS- dependent manner (19). FIG. 5 illustrates the protection of αKGDH by αT and γT present during exposure to the peroxynitrite (RNS)-generating compound SIN-1. A 400 μM concentration of SIN-1 was sufficient to diminish αKGDH activity by approximately 50% in one hour. Under these conditions of nitrative stress, the αKGDH activity varied in a biphasic manner with respect to tocopherol concentration. At highertocopherol concentrations, the reaction medium became grossly turbid so that the apparent loss of enzyme activity might reflect a nonspecific physical consequence of the extreme lipid content. At all concentrations tested, γT was more protective than αT when tested in side-by-side comparisons. Maximal protection was observed at about 1 μM tocopherol in the case of both αT and γT (FIG. 5). The maximal protection by γT was approximately 2.5 times greater than the maximal protection afforded by αT. At concentrations near 100 nM, γT was approximately 5 times more protective than the corresponding concentration of αT. Moreover, 50-100 nM of γT offered as much protection as 1-10 μM of αT. Thus, γT may be as important (or more important) an antioxidant as αT during nitrative stress, despite the lower intrinsic concentration of γT in most mammalian tissue.

### EXAMPLE 4

Demonstration of Neuronal Protection Against Nitrative Stress by γ-Tocopherol

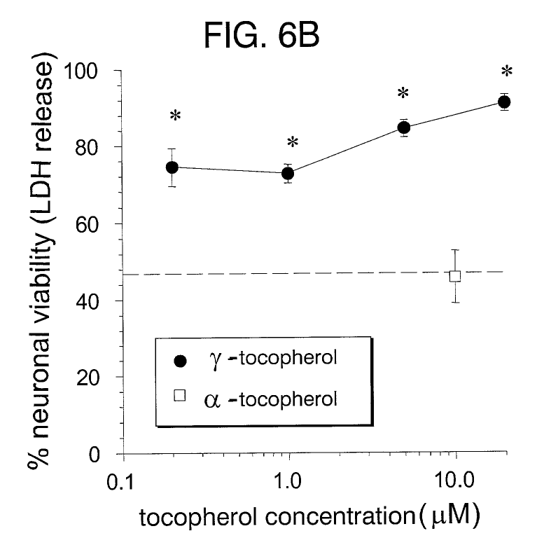

γ-Tocopherol was further assessed for protective ability in a neurotoxicity assay where nitrative stress was imposed. Primary cortical rat neurons were cultured and treated overnight with αT or γT at 10 μM. Media was removed and cells were then washed and medium was replaced with fresh, tocopherol-free medium. Neurons were then exposed to the nitric oxide-generating compound S-nitrosoprusside (SNP) at 10 μM (initial concentration) for 24 hours and toxicity evaluated by release ofthe marker enzyme lactate dehydrogenase (LDH). As illustrated in FIGS. 6A and 6B, γT significantly protected neurons from SNP at a concentration of 200 nM while α-tocopherol was ineffective at a concentration of 10 μM. Thus, in this particular cytotoxicity assay, γT may be orders of magnitude more protective against RNS stress than is αT.

### REFERENCES

The following references are incorporated in pertinent part by reference herein for the reasons cited.

1. Bieri, J. G., Evarts, R. P. Gamma tocopherol: Metabolism, biological activity and significance in human vitamin E nutrition. J. Clin. Nutr. 27: 980-985; 1974.

2. Lehmann, J.; Martin, H. L.; Lashley, E. L.; Marshall, M. W.; Judd, J. T. Vitamin E in foods from high and low linoleic acid diets. J. Am. Diet. Assoc. 86, 1208-1216; 1986.

3. Handelman, G. J.; Machlin, L. M.; Fitch, K.; Weiter, J. J.; Dratz, E. A. Oral α-tocopherol supplements decrease plasma γ-tocopherol levels in humans. J. Nutr. 115: 807-813; 1985.

4. Traber, M. G.; Burton, G. W.; Hughes, L.; Ingold, K. U.; Hidaka, H.; Malloy, M.; Kane, J.; Hyams, J.; Kayden, H. J. Discrimination between forms of vitamin E by humans with and without genetic abnormalities of lipoprotein metabolism. J. Lipid Res. 33, 1171-1182; 1992.

5. Cooney, R. V.; Franke, A. A.; Harwood, P. J.; Hatch-Pigott, V.; Custer, L. J.; Mordan, L. J. γ-tocopherol detoxification of nitrogen dioxide: Superiority to α-tocopherol. Proc. Natl. Acad. Sci. USA. 90: 1771-1775, 1993.

6. Christen, S.; Woodall, A. A.; Shigenaga, M. K.; Southwell-Keely, P. T.; Duncan, M. W.; Ames, B. N. γ-tocopherol traps mutagenic electrophiles such as NOx and complements α-tocopherol: Physiological implications. Proc. Natl. Acad. Sci. USA 94: 3217-3222; 1997.

7. Goss, S .P. A.; Hogg, N.; Kalyanaraman, B. The effect of α-tocopherol on the nitration of γ-tocopherol by peroxynitrite. Arch. Biochem. Biophys. 363: 333-340; 1999.

8. Dillard, C. J.; Gavino, V. C.; Tappel, A. L. Relative antioxidant effectiveness of α-tocopherol and γ-tocopherol in iron-loaded rats. J. Nutr. 113: 2266-2273; 1983.

9. Saldeen, T.; Li, D.; Mehta, J. L. Differential effects of alpha- and gamma-tocopherol on low-density lipoprotein oxidation, superoxide activity, platelet aggregation and arterial thrombogenesis. J. Am. Coll. Cardiol. 34: 1208-1215; 1999.

10. Tran, K.; Chan, A. C. Comparative uptake of alpha- and gamma-tocopherol by human endothelial cells. Lipids 27: 38-41; 1992.

11. Hensley, K.; Maidt, M. L.; Yu, Z. Q.; Sang, H.; Markesbery, W. R.; Floyd, R. A. Electrochemical analysis of protein nitrotyrosine and dityrosine in the Alzheimer brain reveals region-specific accumulation. J. Neurosci. 18: 8126-8132; 1998.

12. Smith M A, Harris P L R, Sayre L M, Beckman J S, Perry G. Widespread peroxynitrite-mediated damage in Alzheimer's disease J. Neurosci 17: 2653-2657; 1997.

13. Tohgi H, Abe T, Yamazaki K, Murata T, Ishizaki E, Isobe C. Remarkable increase in cerebrospinal fluid 3-nitrotyrosine in patients with sporadic amyotrophic lateral sclerosis. Ann Neurol 46:129-131; 1999.

14. Liberatore G T, Jackson-Lewis V, Vukosavic S, Mandir A S, Vila M, McAuliffe W G, Dawson V L, Dawson T M, Przedborski S. Inducible nitric oxide synthase stimulates dopaminergic neurodegeneration in the MPTP model of Parkinson disease. Nat Med 5: 1403-1409; 1999.

15. Sano M; Ernesto C; Thomas R G; Klauber M R; Schafer K; Grundman M; Woodbury P; Growdon J; Cotman C W; Pfeiffer E; Schneider L S; Thal L J. A controlled trial of selegiline, alpha tocopherol, or both as treatment for Alzheimer's disease. The Alzheimer's Disease Cooperative Study. N Engl. J. Med. 336: 1216-1222; 1997.

16. Grundman, M. Vitamin E and Alzheimer disease: The basis for additional clinical trials. Am. J. Clin. Nutr. 2000 (suppl) 630S-636S; 2000.

17. Shintani D, DellaPenna D. Elevatin the vitamin E content of plants through metabolic engineering. 282: 2098-2100; 1999.

18. Hensley, K.; Williamson, K. S.; Maidt, M. L.; Gabbita, S. P.; Grammas, P.; Floyd, R. A. Determination of biological oxidative stress using high performance liquid chromatography with electrochemical detection (HPLC-ECD). J. High Res. Chromatogr. 22: 429-437; 1999.

19. Park, L. C.; Zhang, H.; Sheu, K. F.; Calingasan, N. Y.; Kristal, B. S.; Lindsay, J. G.; Gibson, G. E. Metabolic impairment induces oxidative stress, compromises inflammatory responses, and inactivates a key mitochondrial enzyme in microglia. J. Neurochem. 72: 1948-1958; 1999.

1. A method of preventing, delaying or reversing symptoms and consequences of neurodegenerative disease which comprises the administration to an appropriate subject of an effective amount of gamma tocopherol.

2. The method of claim 1 wherein the gamma tocopherol is pure.

3. The method of claim 1 wherein the gamma tocopherol is part of a tocopherol mixture.

4. A method of delaying or preventing the symptoms and consequences of neurodegenerative disease which comprises the administration to a subject of an effective amount of at least one desmethyl tocopherol.

5. The method of claim 1 or 4 wherein the neurodegnerative disease is Alzheimer's disease, Parkinson's disease or amylotrophic lateral sclerosis.

6. The method of claim 1 or 4 further defined as comprising administration of at least one additional antioxidant.

7. The method of claim 1 or 4 where the tocopherol used is a mixture of isomers.

8. The method of claim 1 or 4 where the tocopherol is isolated from natural products.

9. The method of claim 1 or 4 where the tocopherol is synthetically prepared.

10. The method of claim 1 or 4 where the tocopherol is administered as a prodrug.

11. The method of claim 1 or 4 where the tocopherol is administered as a water-soluble ester.

12. The method of claim 1 or 4 wherein the amount of tocopherol administered is from about 100 to about 400 mg/day.

13. The method of claim 1 or 4 wherein the subject is a patient showing early symptoms of neurological disease.

14. The method of claim 1 or 4 where the subject has a history of familial neurological disease.
